(19) United States
(12) Patent Application Publication
(10) Pub. No.: US 2022/0133159 A1
(43) Pub. Date: May 5, 2022

(54) Non-Invasive, Continuous, Accurate and Cuff-Less Measurement of Blood Pressure and Other Cardiovascular Variables by Pulse Wave Acquisition and Analysis Using Non-Invasive Sensors

(71) Applicant: THE REGENTS OF THE UNIVERSITY OF MICHIGAN, Ann Arbor, MI (US)

(72) Inventors: Sardar Ansari, Ypsilanti, MI (US); Kenn Oldham, Ann Arbor, MI (US); Kevin R. Ward, Superior Township, MI (US); Kayvan Najarian, Northville, MI (US); Lu Wang, Ann Arbor, MI (US)

(21) Appl. No.: 17/514,939

(22) Filed: Oct. 29, 2021

Related U.S. Application Data
(60) Provisional application No. 63/107,982, filed on Oct. 30, 2020.

(51) Int. Cl. A61B 5/021; A61B 5/00

(52) U.S. Cl. CPC A61B 5/02116; A61B 5/02125; A61B 5/6831; A61B 5/6826; A61B 5/6822; A61B 5/6824; A61B 5/6828; A61B 5/6819; A61B 5/6823; A61B 5/6829

(57) ABSTRACT

A method for noninvasively measuring hemodynamic variables of a person includes physically configuring a sensor to measure the pulse of a person. The sensor generates a pulse waveform indicative of the pulse of the person. A processor obtains the pulse waveform from the sensor and the processor determines a reflection coefficient and reflection delay between an incident and a reflected wave, from which the processor determines the hemodynamic variables of the person from the reflection coefficient and the reflection delay.

### CROSS-REFERENCE TO RELATED APPLICATIONS

This application claims priority to U.S. Provisional Patent Application No. 63/107,982, filed Oct. 30, 2020, which is hereby incorporated by reference in its entirety.

### STATEMENT OF GOVERNMENT SUPPORT

This invention was made with government support under CMMI1562254 awarded by the National Science Foundation and TR002240 awarded by the National Institutes of Health. The government has certain rights in the invention.

### FIELD OF THE DISCLOSURE

The present disclosure relates to cardiovascular monitoring and, more particularly, to techniques for determining hemodynamic variables from wearable devices.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Hemodynamic variables such as blood pressure (BP) and vascular resistance (VR) are key physiological indicators of health. They reflect the overall physical wellbeing of an individual. For example, hypertension (i.e., high BP) is the gateway disease for more serious medical conditions such as diabetes, ischemic heart disease, heart failure, stroke, and chronic kidney disease, to name a few. According to the most recent reports, more than 46% of the US population suffers from hypertension. About 20% of those suffering from hypertension are unaware that they have the condition. For most adults in the US, the only time their BP is recorded is during their primary care clinician's office visits. However, many studies have shown substantial differences between in-clinic and in-home measurements. This is due to the white coat effect (overestimation of BP in the clinic) and masked hypertension (underestimation of BP in the clinic), leading to an inaccurate diagnosis of hypertension. In addition, the infrequent measurements made during clinic visits may result in delayed diagnosis of the disease, inhibiting preventive and corrective measures for hypertension and subsequent health conditions.

Despite the importance of BP as a major health indicator in a large range of medical conditions, current clinical practice uses >100-year-old techniques (auscultatory and oscillometric methods) to measure BP. There is a great need for more versatile BP monitors that can be incorporated into smart wearable devices to track BP accurately and continuously without interfering with daily physical activity. While this need is widely recognized, the proposed solutions suffer from major shortcomings. Most importantly, all of the existing technologies that have been independently verified require a pressure cuff and/or frequent recalibrations.

Cardiac output (CO) is one of the most important indicators of cardiac health and hemodynamic balance. It measures the amount of blood that the heart supplies to the organs and cells, indicating how much oxygen and nutrients are delivered and how much waste is removed. Assessments of CO are frequently used to guide diagnosis and treatment in critical care settings. For example, cardiogenic and septic shock may both present with low BP and similar presenting symptoms but the pathophysiology of the two diseases is markedly different with one cardiogenic having a low CO and septic shock often presenting a high CO. The treatment required for the two diseases is different and therefore the identification of CO of a patient may be useful for diagnosing diseases. In addition to acute conditions, changes in CO also play a pivotal role in the development of chronic conditions. For example, heart failure is accompanied by a reduction in CO. Thus, continuous, non-invasive measurement of CO may assist the diagnosis and treatment of heart failure. However, CO is only measured in acute settings because there is no reliable method for measuring it using wearable devices.

Thus, there is a need for effective solutions for continuous, accurate, and portable measurement of hemodynamic variables to properly monitor and diagnose diseases in patients.

### SUMMARY

The present techniques address shortcomings of current hemodynamic monitoring methods by enabling continuous monitoring of a person's BP and other cardiovascular parameters such as CO, VR, and arterial stiffness. Further, the disclosed methods and devices are non-invasive and require less sensor alignment and calibration than traditional methods for hemodynamic monitoring. The devices may be worn by a person for hours, or days for continuous monitoring of the person's BP and other cardiovascular variables.

In accordance with an example, a device for non-invasively measuring hemodynamic variables includes a sensor configured to sense a pulse of a person and to generate signals indicative of the pulse of the person. A support structure is physically coupled to the sensor to physically support the sensor during operation of the sensor. The device further includes a processor in communication with the sensor with the processor being configured to collect the signals indicative of the pulse of the person from the sensor, determine, from the signals, a waveform representative of the pulse, perform signal processing on the waveform to identify features of the waveform, and perform processing on the features to determine the hemodynamic variables of the person.

### BRIEF DESCRIPTION OF THE DRAWINGS

FIG. 1 is a diagram illustrating how various hemodynamic parameters (i.e., arterial pressure, CO and VR) are related to each other and how to determine one hemodynamic parameter from the other two.

FIG. 2A is a diagram of a pulse monitoring device employed as a ring sensor to be worn on a finger of a person, depicting one possible embodiment of the proposed system.

FIG. 2B is a diagram of an embodiment of an electrical layout of the ring sensor of FIG. 2A.

FIG. 3 is a plot of a sample waveform obtained using a polyvinylidene fluoride based sensor, such as the sensor of FIG. 2A, obtained from a swine.

FIG. 4 is a plot of pulse waveforms generated by a PVDF sensor at different levels of BP and systemic VR (SVR) during pulmonary artery catheterization.

FIG. 5 is a flow diagram of an embodiment of a method for determining the BP, CO, and VR of a person.

FIG. 6 includes multiple plots of signals versus time for animals during baseline, the norepinephrine maneuver to increase BP and VR, and the hyperventilation maneuver to decrease BP and VR with white dots marking the identified incident (forward) waves and black dots marking the identified reflection (backward) waves.

FIG. 7 is a plot of arterial BP for different sample times measured in swine showing top and bottom boundaries correspond to systolic and diastolic pressures, respectively and red and green dots corresponding to estimated systolic and diastolic pressures, respectively.

FIG. 8A is a plot of estimated systolic BP versus the BP as measured by an invasively systolic pressure measurement.

FIG. 8B is a plot of estimated diastolic BP versus the BP as measured by an invasively diastolic pressure measurement.

FIG. 9 is a block diagram of an example signal processing device to determine hemodynamic variables such as BP from measured pulse signals, in accordance with an example herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DESCRIPTION

Existing techniques that attempt to measure BP continuously employ cuff-less estimation of BP using the principle of pulse wave velocity (i.e., the speed by which blood travels through the arteries). The existing technologies suffer from multiple shortcomings that prohibit their use in commercially-available wearable monitoring devices. For example, current cuff-less technologies require two sensors that are far apart on different regions of the body to measure the pulse travel time which increase complexity and complicates sensor placement. Further, the sensors and associated fixtures need to be re-calibrated frequently and the method often makes inaccurate assumptions about certain physiological factors (e.g., VR), leading to imprecise estimates of BP. The disclosed approach provides a practical tool to measure BP accurately, continuously, and conveniently.

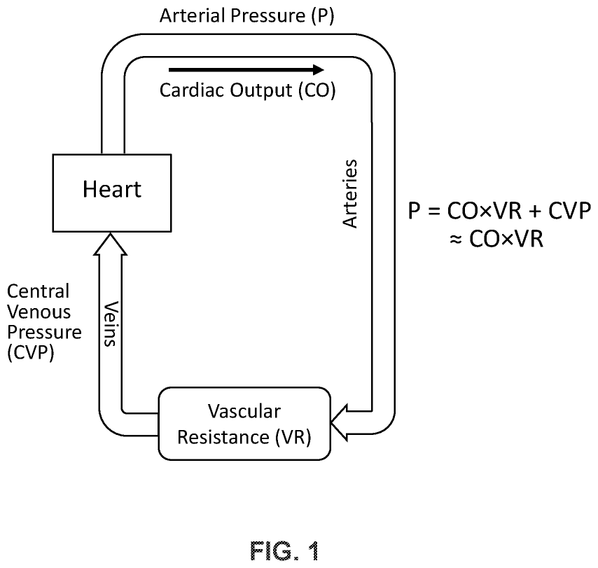

A small cuff-less wearable device capable of accurately measuring hemodynamic parameters such as BP and CO could have a significant impact on overall public health by enabling long-term monitoring of individuals' cardiovascular health at a large scale over the course of decades. The data obtained by such a device could help in early identification of individuals at risk of hypertension. In addition, such a technology allows for targeted monitoring of patients for whom BP fluctuations may be indicative of critical states such as hemodynamic instability. By measuring BP and CO, we can also calculate VR. Vascular resistance is another hemodynamic variable that characterizes the function of the cardiovascular system. FIG. 1 is a diagram illustrating how VR can be determined from arterial pressure and CO. By measuring the arterial pressure, P, and determining the CO, the VR can be determined by

P=CO×VR+CVP≈CO×VR. EQ. (1)

CVP is the central venous pressure and is typically negligible and can be estimated to be about 0 mmHg. Therefore, VR can be estimated from CO and P according to EQ. (1). As a result, the proposed technology will provide a more holistic approach towards monitoring of the cardiovascular health by determining multiple hemodynamic variables.

One embodiment of sensors that can be used to measure the pulse wave to estimate hemodynamic variables, such as BP and VR, may be made of Polyvinylidene fluoride (PVDF) materials. PVDF-based sensors have been widely studied and employed in applications such as energy harvesting, as transducers, and as actuators, among other uses and applications. PVDF materials are used in a broad range of applications due to their shape adaptability, flexibility, inexpensive cost and easy fabrication. Further, due to the fact that PVDF materials are comfortable to wear and easy to use, they have gained popularity in health monitoring applications such as pulse rate monitoring, respiration monitoring, sleep monitoring, and body motion monitoring.

PVDF sensors typically include a pair of plate-shaped electrodes that cover a piezoelectric layer of a polymer. This forms a capacitive element with the polymer acting as dielectric material. The charge on the capacitive element changes under different applied mechanical stresses (e.g., a bend, torque, compression, etc.). PVDF sensors can be implemented to measure pulses of a patient and pulse waves indicative of the pulses may then be analyzed to determine the hemodynamic variables such as BP of the patient. The method disclosed herein uses analytical algorithms to continuously monitor these hemodynamic variables using non-invasive cuff-less sensors. The sensors employed are small, inexpensive, and are low-power, which allows for longer operating times and requires less maintenance of the sensors. The described sensors may include one or more of optical sensors (e.g., for photoplethysmography), force based sensors (e.g., piezoelectric, piezoresistive), electrical based sensors (e.g., impedance), or ultrasonic sensors, among others. In embodiments, a plurality of sensors of any combinations of the types of sensors may be employed for performing the method described herein. A sensor apparatus may be wrapped around the wrist or finger, for example like a watch or a ring, or a sensor apparatus may be placed on or around any other location on the body where a pulse can be sensed such as arm, thigh, ankle, toe, temple, nose, chest or neck, to measure the pulsatile flow of blood with each heartbeat. A physical support structure is physically coupled to the sensor or sensors to hold the sensor in place during operation of the sensor. Examples of potential support structures include a band for a watch or ring, or a bridge structure such as in glasses. The support structure may be any physical structure capable of physically supporting a sensor during operation of the sensor for detecting a pulse as described herein. The sensor(s) generate a waveform, and information about the cardiovascular system, including BP and VR, can be determined from the generated waveform. By estimating multiple hemodynamic variables, the system also increases the accuracy of individual estimations. For example, the estimated VR can be used to enhance the approximation of BP, as is described below. In addition, the estimation of BP and VR would enable us to calculate blood flow, CO and stroke volume.

In examples, the methods disclosed include signal processing and machine learning tools that analyze features of the generated waveforms to measure hemodynamic parameters continuously and accurately. The methods include decomposing a pulse waveform into incident and reflected pulse waveforms. Features of the incident and reflected waveforms may then be determined. For example, a maximum incident pulse amplitude, maximum reflected pulse amplitude, reflected pulse time delay, minimum reflected pulse amplitude value, minimum incident pulse amplitude value, and other features of the pulses may be determined. The features of the reflected pulse, relative to the incident pulse, may then be used to estimate hemodynamic parameters such as BP of a person being monitored by a sensor or sensor apparatus. Multiple methods for decomposing the waveform into the incident and reflected waveforms include deconvolution, echo processing, frequency filtering, and other methods are disclosed herein. Further, a reflection coefficient may be determined form the features of the incident and reflected pulse waveforms and the hemodynamic variables such as VR may be determined from the reflection coefficient. Additionally, CO of a person may be determined from the features of the incident waveform, features of the reflected waveform, and reflection coefficient.

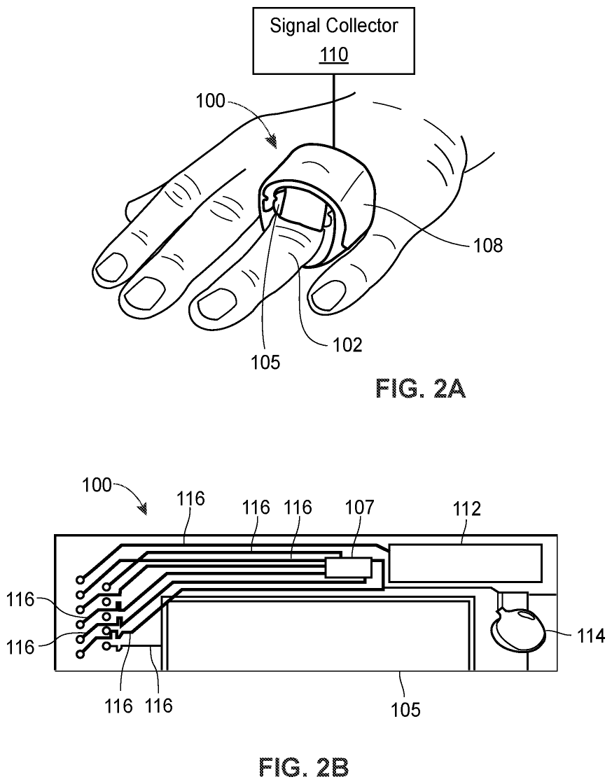

FIG. 2A is a diagram of a pulse monitoring device employed as a wearable sensor, in this example a ring sensor 100 to be worn on a finger 102 of a person. FIG. 2B is a diagram of an embodiment of an electrical and mechanical layout of the ring sensor 100. Referring simultaneously to FIGS. 1A and 1B, the ring sensor 100 includes a pulse sensor 105, and a physical support structure in the form of a band 108 physically coupled to the pulse sensor 105. The ring sensor further includes an electrical ground 112, a power source 114, circuitry 107, and electrical lead lines 116. The band 108 is configured to hold the pulse sensor 105 in a position for the pulse sensor 105 to measure pulses of the person. The band 108 may include an elastic band, a Velcro band, a rubber band, medical tape, a buckle, strands for tying, or another band or material capable of holding the pulse sensor 105 in a position. The ring sensor 100 may further include a tightening mechanism that allows for adjusting the pressure of the pulse sensor 105 on the skin of the person. For example, a screw may be a tightening mechanism that allows a user to increase or decrease the pressure of the pulse sensor 105 against the skin of the person. In another embodiment, a pressure/tension adjustment mechanism may be used that incorporates a bladder containing a gas or fluid, or a mechanical actuator such as those made of piezoelectric material. The ring sensor 100 may be worn at a variety of different sites on the body of a person such as on a finger, a wrist, an ankle, or another body part or region of the body. The circuitry 107 may include an amplifier, low-pass filter, high-pass filter, bandpass filter, notch filter, amplifier, integrator, or another electrical element for performing signal conditioning and processing. Further, the circuitry 107 may include an integrated circuit, independent circuit elements (e.g., resistors, capacitors, operational amplifiers, etc.) an FPGA, or another circuit element. The ground 112 creates a contact between skin of the person and acts as a grounding electrode which may be used to reduce noise due to the power source 114. In embodiments, the pulse sensor 105 may be a PVDF sensor that does not need to be powered. A PVDF sensor can be a passive sensor that converts mechanical tension into an electrical current without requiring any external power supply. Therefore, in embodiments, the power source 114 may provide power to the circuitry 107. Further, the ring sensor 100 may not include the circuitry 107, and any circuitry other than the pulse sensor 105 may be in an external device and therefore, in embodiments, the ring sensor 100 may also not include or require any power source. In embodiments, any circuitry for collecting and processing the signal from the pulse sensor 105 may include a computer, tablet, phone (e.g., as performed by an app on a phone) server, cloud, mobile device, or other computational device.

In embodiments, a signal collector 110 may be in communication with the ring sensor 100. The signal collector 110 collects signals indicative of measured pulses from the ring sensor 100. The signal collector 110 may perform further signal processing on the collected signals, and/or the signal collector 110 may provide the collected signals to another computer, or network for further processing of the signals. In embodiments, any or all of the circuitry 107 may be included in the signal collector 110. The signal collector 110 may be in communication with the ring sensor 100 through wired or wireless communicative means. For example, the ring sensor 100 may include an IO chip (e.g., having one or more processors and one or more memories) for providing communications with other computers, networks, and devices. The signal collector 110 may be a data acquisition system or a component of a data acquisition system. In embodiments, the signal collector 110 may collect the signals from the ring sensor 100 and provide data indicative of the collected signals to a network. A person, at a remote location, may then use the disclosed methods to analyze the data to determine a BP, VR and/or CO of a person. For example, a user wearing the ring sensor 100 may be at home, a grocery store, or driving in a car, and a medical professional stationed at a hospital may access data indicative of the pulses of the user. The medical professional can then determine, monitor, and/or log the BP, VR and/or CO of the person continuously in real time. In another embodiment, the data can be analyzed by the sensor or a stand-alone device or algorithm and stored for future use. For example, the extracted hemodynamic variables can be aggregated and used at a later date to assess the subject's health status. This information can be provided to the user through an application or website to give them feedback about their health.

The methods for measuring the pulses of a person as described herein, may depend on the morphology of generated pulse waveforms, which is determined by systemic cardiac factors that affect blood flow such as vasculature resistance, cardiac health, etc. Therefore, the morphology of pulse waveforms obtained by the described method and devices is independent of the measuring site of the sensor. As such, the disclosed methods of determining hemodynamic parameters such as BP do not depend on the absolute amplitudes of the waveforms and no calibration of the sensors is required when it is removed and replaced. Further, typical frequency ranges of cardiac pulse cycles do not vary substantially so dynamic effects due to cardiac activity are typically relatively uniform across a waveform.

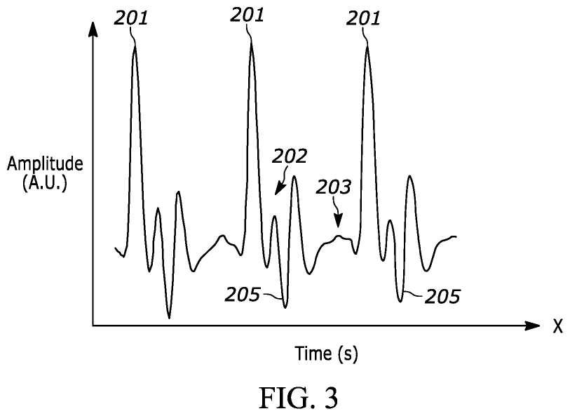

FIG. 3 is a plot of a sample waveform obtained using a PVDF based sensor from a swine. In the swine model data further discussed below, the sensor was placed distally over the radial artery on the swine's foreleg adjacent to the hoof. The placement of the sensor allowed for monitoring of pulses through the swine's smallest accessible radial artery, which is analogous to monitoring pulses in the human wrist. The PVDF sensor acts as a high-pass filter because of the input impedance and internal capacitance of both the PVDF sensor and a data acquisition system configured to collect data from the PVDF sensor, with the data being indicative of a pulse waveform. An amplification circuit, such as may be included in the circuitry 107 of FIG. 2B, produces a transfer function operation to convert a strain of the PVDF sensor into a voltage. In embodiments described herein, the transfer function had a zero at the origin and two real poles. The dominant pole-zero dynamics of the transfer function caused the amplification circuit to perform as a high-pass filter with a cutoff frequency higher than the signal frequency, such that the amplification circuit behaved as a differentiator in the frequency range of the cardiac cycles. Therefore, the generated signal indicative of the measured pulses is approximately the derivative of the pressure waveform on the PVDF sensor with respect to time at the measurement site. The pulse waveform presented in FIG. 3 shows pulse peaks 201, dicrotic notches 205, a first reflected wave 202, and second reflected wave 203. Each of the peaks 201, notches 205, and first and second reflected waves 202 203 are periodic according to the periodicity of the cardiac cycles. As previously discussed, the features (e.g., amplitude, overall shape, temporal position, etc.) of the peaks and troughs in the waves 201, 202, 203, and 205 depend on the monitored arterial branch and the distance of the sensor along the arterial branch from the heart. In addition, the peaks and troughs vary with changes in the hemodynamic variables such as BP, CO and VR. The dicrotic notches 205 are created by the closure of the aortic valve between systole and diastole. In implementations, the dicrotic notches 205 may be used for determining one or more hemodynamic variables. The first reflected waves 202 are analyzed to determine the hemodynamic variables such as BP, CO, cardiac index (CI), VR, and SVR of a person being monitored by a sensor as described further herein. The second reflected waves 203 may be analyzed using the same techniques described with reference to the first reflected waves 202 to determine the hemodynamic variables. The first reflected waves 202 and second reflected waves 203 reflected from different parts of the body or a vascular system allowing for the measurement of hemodynamic variables at different parts of the body simultaneously, using a single sensor. Further, the first reflected waves 202 and second reflected waves 203 may provide better/more accurate measurements of hemodynamic variables depending on the location of the measurement of the pulse and the origins (i.e., location in the body) of the first and second reflected waves 202 and 203. Additionally, more than two reflected pulses may be measured and hemodynamic variables may be determined using any of the reflected pulses.

The elastic modulus of large arteries is a function of the intra-arterial pressure,

E(P)=E₀e^{αP}. EQ. (2)

P is the pressure inside of the artery, a is an artery dependent constant and E₀ denotes the elastic modulus when arterial pressure is zero. The Moens-Kortweg formula may be used to model the artery as an elastic tube to relate the artery elastic modulus with pulse wave velocity (PWV) by

$$PWV = \sqrt{\frac{h E}{2 r ρ}} . \qquad EQ . ( 3 )$$

In EQ. 3, h and r are the artery thickness and radius, respectively, and ρ is the density of blood. Using EQs. (2) and (3), we can find the relationship between arterial pressure and PWV as

$$P = {α}^{- 1} log ( \frac{2 r ρ}{h {E}_{0}} ) + 2 {α}^{- 1} log ( P W V ) . \qquad EQ . ( 4 )$$

The major contributor to arterial pressure, P, is the PWV. We derive the PWV from a pulse transit time in an arterial branch. The pulse transit time of some noninvasive BP measurement techniques is measured by employing two sensors at two different anatomic sites, for example, by employing an electrocardiograph sensor and a photoplethysmograph sensor placed on a finger. As described herein, the pulse transit time of the current disclosed system and methods may also be measured by estimating the time delay between a forward (incident) pulse wave and a reflection wave of the incident pulse wave on a generated pulse waveform, such as the waveform of FIG. 3. Therefore, is determined herein as the inverse of the time delay between the forward incident pulse wave and the backward reflection wave in the pulse waveform, which correlates with the arterial BP. Calculating the PWV from the time delay of the reflected waveform allows for the use of a single sensor as opposed to traditional pulse transit time methods which require multiple sensors. Therefore, calculating the PWV as described herein, simplifies required sensing and processing hardware, and reduces required processing for determining hemodynamic variables of a person.

The main contributor to the first term on the right-hand side of Equation (4) is the VR, R, which is mainly determined by artery radius, r. According to the Hagen-Poiseuille equation, the VR may be calculated as

$$R = \frac{8 L η}{π {r}^{4}} . \qquad EQ . ( 5 )$$

L and η are the length of the artery and blood viscosity, respectively which are both relatively constant and can be calibrated over the lifetime of a person. Arterioles and capillaries have small radii and are therefore the predominant contributors to VR. The length of the artery, L, is a constant and the variations in η are negligible compared to variations in the r⁴ term, which can change with the activation of the smooth muscle around arterioles. Therefore, changes in the VR, R, are mainly determined by variations of the arteriole radius. The body uses changes in the VR, through increasing and decreasing the artery radius (vasoconstriction and vasodilation, respectively), to maintain homeostasis. Through controlling R, vasoconstriction and vasodilation maintain BP and CO within a set of ranges. Changes in R impact BP, as seen in Eq. (4), and should be taken into account when estimating BP of a person, although, existing methods for estimating BP assume a constant value for r which leads to estimation error.

Pressure wave reflections are generated when there is a change in the radius of the vessels as pulse travels through the distal arteries, arterioles and capillaries. The reflections are caused by the change in the characteristic impedance of the vessel, resulting in a backward propagation of a reflected pulse wave. The reflection coefficient, F, determines the amplitude of the backward wave. It can be calculated by

$$Γ = \frac{{Z}_{T} - {Z}_{0}}{{Z}_{T} + {Z}_{0}} = \frac{{A}_{r}}{{A}_{i}} . \qquad EQ . ( 6 )$$

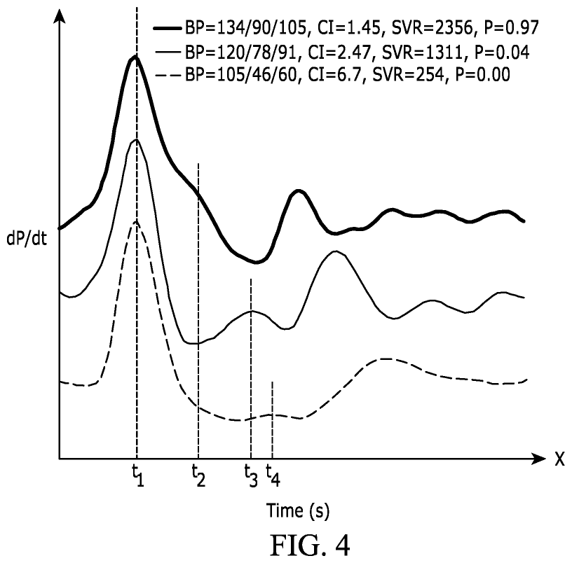

The characteristic impedances in the proximal and distal (terminal) arteries are denoted by Z₀ and Z_{T}, respectively. Vasoconstriction leads to an increase in Z_{T} and the amplitude of the reflected wave, Aᵣ. Further, the reflection coefficient may be calculated as the ratio of the amplitude of the reflected wave Aᵣ divided by the amplitude of the incident wave Aᵢ. Therefore, the amplitude of the reflection wave, as measured by a sensor, correlates with VR; hence, the reflected pulse wave contains information regarding both PWV and r that can be used to estimate P using EQ. 4. FIG. 4 is a plot of pulse waveforms generated by a PVDF sensor at different levels of BP and SVR during pulmonary artery catheterization. The waveforms of FIG. 4 demonstrate how the time delay between incident (forward) and reflected (backward) pulse waves increases as BP decreases. The waveforms of FIG. 4 are time derivatives of pressure as measured by PVDF sensors. The curves of FIG. 4 demonstrate how the amplitude of a reflected wave decreases as SVR also decreases. The peak amplitude of each incident pulse wave occurs at time t₁. The peak amplitude of the reflected waves occurs at time t₂ for SVR=2356, at t₃ for SVR=1311, and at t₄ for SVR=254. It is evident from the curves of FIG. 4 that as SVR decreases, the reflected amplitude decreases and the reflected pulse delay increases. The peak to the right of each of the reflected wave peaks is indicative of the dicrotic notch in each curve.

In an example, the disclosed detection device and methods were performed using animals as subjects. A PVDF sensor was placed on the foreleg of five swine and maneuvers were performed to induce large amounts of variation in the BP of the swine causing BP ranging from 35/20 to 260/150 mmHg. A high average correlation of 0.94 (i.e., ranging from 0.90 to 0.98) was found between the true and estimated pressure. The true BP was determined by a clinical-grade invasive measurement of intra-arterial BP while the estimated pressure was determined using the PVDF sensor in the form of a sensor ring.

In addition to the estimation of BP and VR, the proposed methods can also be used to estimate CO and cardiac index by using the hydraulic version of Ohm's law:

$$Q = \frac{Δ P}{R} \approx \frac{{P}_{a}}{R} , \qquad EQ . ( 7 )$$

where Q denotes the CO, and ΔP is the gradient between the mean arterial and mean right atrial pressures. The right atrial pressure is often close to zero, therefore the CO can be approximated as the ratio between the mean arterial pressure (MAP), Pₐ, and R. As previously discussed, R may be determine from the relative amplitudes of the incident and reflected pulses, and Pₐ may be determined from both the relative amplitudes and the delay of the reflected pulse, after which CO may then be determined according to EQ. 7. Therefore, decoupling the forward and backward waves and determining properties of the waves (i.e., relative amplitudes and time delay) can lead to the simultaneous accurate estimations of BP, CO, and VR.

The shape of the pulse wave changes as it travels through an arterial tree. The shape change depends on the elasticity and the mechanical dynamics of the arteries, both of which change with age and arterial diseases. As such, a transfer function between the forward and backward waves, which can be calculated after decoupling of the forward and backward waves, can characterize the arterial stiffness and other factors of the arterial health of the subject. In one example, the transfer function may be determined by performing a Fourier transform of each of the incident and reflected waves, and then dividing to determine the transfer function. The transfer function represents the mechanical dynamics of the arterial system of a person. Therefore, the described monitoring devices and methods are able to characterize patient diseases beyond the measurement of hemodynamic parameters and further allow for long-term monitoring of chronic diseases such as arteriosclerosis and peripheral artery disease. In addition, characterization of the coefficients in EQ. (4) allow for estimation of α, h, E₀, and ρ, providing additional information about the arteries and the cardiovascular system. The additional information enables the systems and methods to characterize the mechanical function of arteries and the cardiovascular system of a person.

The propagation of the pulse wave through the arterial tree leads to more than one reflection as seen in FIG. 3 with the appearance of at least two reflection waves 202 and 203. These reflection waves may be generated from different anatomical sites (e.g., the brain and a lower body limb such as an arm, etc.). Hence, they can be used to distinguish between changes in VR that happen within different organs and sections of the cardiovascular system. By doing so, the disclosed approach may not only provide an estimate for the overall VR (i.e., systemic vascular resistance), but also the various constituents of VR arising from the microcirculation and small arteries in different parts of the body. This allows for better characterization of the changes in the VR and the physiological response of the body to various stimuli. For example, a reflection wave generated when the incident wave reaches the vasculature of the brain may enable estimation of the cerebral vascular resistance, while reflection waves generated at peripheral organs may enable estimation of VR in other parts of the body.

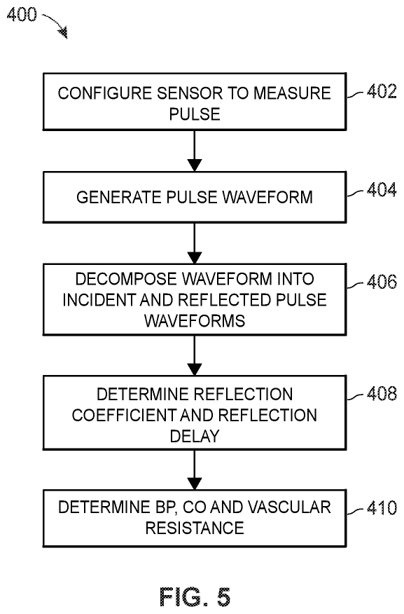

FIG. 5 is a flow diagram of an embodiment of a method 400 for determining the BP, CO, and VR of a person. The method 400 of FIG. 5 may be performed by the ring sensor 100 of FIGS. 1A and 1B. The method 400 includes physically positioning a sensor to measure the pulse of a person (block 402). In embodiments, the sensor may be a PVDF sensor, an optical sensor (e.g., for photoplethysmography), a force based sensor (e.g., piezoelectric, piezoresistive), an electrical based sensor (e.g., impedance), an ultrasonic sensor, or another type of sensor for sensing the pulse of a person. In embodiments, a plurality of sensors may be physically positioned to measure the pulse of the person. In embodiments, any combinations of types of sensors may be used to monitor the pulse of a person. Further, the sensor may be positioned on a person's wrist, finger, arm, ankle, waist, leg, or another part of the body for measuring the pulse of the person.

The sensor measures the pulses of the person and generates a composite pulse waveform (block 404). The composite pulse waveform includes data indicative of both forward and backward propagating pulses in the form of an incident pulse and reflected pulse, respectively. As previously discussed, the composite waveform may be indicative of more than one backward propagating pulse or wave, any of which may be used to determine hemodynamic variables. For simplicity and clarity, the method 400 will be described with reference to a single reflected pulse. The method further includes decomposing the composite waveform into an incident pulse waveform and a reflected pulse waveform (block 406). In embodiments, decomposing the composite waveform may include performing echo cancelation on the composite waveform. The reflected pulse waveform can be interpreted as an echo of the incident pulse waveform and, therefore, echo cancellation can be used to remove the reflected pulse waveform from the composite waveform resulting in the isolation of the incident pulse waveform. The resultant incident pulse waveform can then be subtracted from the composite waveform to generate the reflected pulse waveform.

In some embodiments, decomposing the composite waveform may include an optimization process wherein an objective function maximizes some measure or measures of smoothness of a transformed version of the incident wave as compared to the reflected wave. For example, the absolute value of the second derivative of the incident or reflection wave can be used as an objective function since it measures the smoothness of the signal. In embodiments, decomposing the composite waveform may include a deconvolution. The reflected waveform may be interpreted as a delayed version of the incident waveform convolved with an impulse response of the arterial system that the incident pulse and reflected pulse are propagating through. As a result, the incident waveform can be reconstructed by deconvolving the reflected waveform from the composite waveform. For deconvolution, a model for the frequency response of the arterial system can be restricted to have a limited number of parameters if necessary.

In other embodiments, the objective function that is optimized can use the frequency contents of the forward and backward waves. Since the backward wave is a filtered version of the forward wave, the two waves have approximately the same frequency contents. It is known that the amplitude of the sum of two waves with the same frequency contents is maximized when the two waves are aligned, i.e., they have the same phase. This fact can be used to find the delay between the forward and backward waves by using an objective function based on some combination of the amplitudes of the forward and backward waves. For example, one can use the amplitude of the Fourier transform of the forward and backward waves to derive an objective function that is maximized when the correct delay is used to separate the waves.

Filtering may be performed on the composite waveform to isolate the reflected pulse waveform and/or the incident pulse waveform. For example, a non-adaptive filtering approach, such as a finite impulse response filter or an infinite impulse response filter, and/or adaptive filtering such as a least mean square filter or a Kalman filter may be used to isolate either of the reflected pulse waveform and/or incident pulse waveform. Further, once one of the incident and/or reflected pulse waveforms have been identified, the other waveform can be determined by subtracting the identified waveform from the composite waveform.

Features of the composite waveform can be directly measured to quantify the location and amplitude of the reflected pulse waveform. For example, signal processing approaches can be used to quantify the location and relative amplitude of reflected waveform peaks in the composite, such as the composite wave shown in FIG. 4. Additionally, signal processing methods (e.g., differentiation, high-pass filtering, etc.) can be used to amplify features associated with the reflected waveforms to better identify the features of the reflected waveforms.

In embodiments, the reflected waves may be identified and their time delay and amplitude may be measured using curve fitting techniques such as Gaussian fitting, polynomial fitting or the use of other basis functions such as sinusoidal functions, wavelet function, or other custom functions

In embodiments, the method may include identifying multiple reflection waves from the reflected pulse waveform. A processor may then determine a hemodynamic variable from the features of the multiple reflected waves.

In embodiments employing multiple sensors for measuring the pulse of a person and various transformations, signal processing techniques, and data manipulation techniques can be used to decompose the composite waveform. For example, principal component analysis, singular value decomposition, independent component analysis, and/or tensor decomposition may be used to generate the incident pulse waveform and the reflected pulse waveform. Further, other transformations such as a Fourier transform, a spectrogram, Stockwell transform, and/or Wavelet transform may be used to create a representation of the composite waveform that allows for the separation of the incident and reflected pulse waveforms.

Decomposing the composite waveform may include performing machine learning techniques such as classification and regression methods. The machine learning techniques and regression methods may include a linear regression, logistic regression, support vector machine, decision tree, random forest, XGBoost, neural network, and k-nearest neighbor, among others. The machine learning technique and/or regression may be applied directly or indirectly (i.e., through extracted features of the waveform) to the composite waveform to estimate BP directly or to separate the incident and reflected pulse waveforms.

Artificial neural networks and deep learning techniques such as different variations of convolutional neural networks and recurrent neural networks may be implemented to identify appropriate filters for estimating the reflected pulse waveform features (e.g., amplitude, time delay, etc.) in the composite signal. Neural network and deep learning approaches can be used to either decompose the incident and reflected waveforms from the composite waveform, or to directly estimate the hemodynamic variables from the patterns associated with, and characteristics of, the incident and reflected waveforms in the composite waveform.

A least absolute shrinkage and selection operator (LASSO) may be used to decompose the composite waveform. The LASSO can be used to model the summation of the incident pulse waveform with a transformed and delayed version of itself. By applying a penalty term to coefficients of the regression, the LASSO technique can be used to identify the location of the backward wave in the composite signal, and to estimate the coefficients for the transformation between the incident and reflected waveforms.

Model-based simulation may be used to decompose the composite waveform. An arterial junction having associated arterial dimensions and properties can be modeled using 1D, 2D or 3D arterial modeling strategies to generate a reflected pulse waveform from a simulated or measured incident pulse waveform profile.

Further, wave duplication may be implemented to decompose the composite waveform. A scaled and delayed copy of the incident pulse waveform can be generated and parameters for pulse amplitude and pulse timing delay can be determined to provide a best fit to measured data to optimize a fit to the composite waveform.

A Windkessel model may be implemented to decompose the composite waveform. For example, a higher-order Windkessel model for lumped parameter cardiovascular dynamics may be used to generate reflected waveform characteristics and properties.

After the composite waveform has been decomposed, the reflection coefficient is determined from various characteristics of the reflected pulse (block 408). For example, the reflection coefficient, F, and VR may be determined by the EQ. 6 as previously described (block 410). The BP is then determined from the delay between the forward and backward waves and EQ. 4. The CO can be determined using BP and VR through EQ. 7.

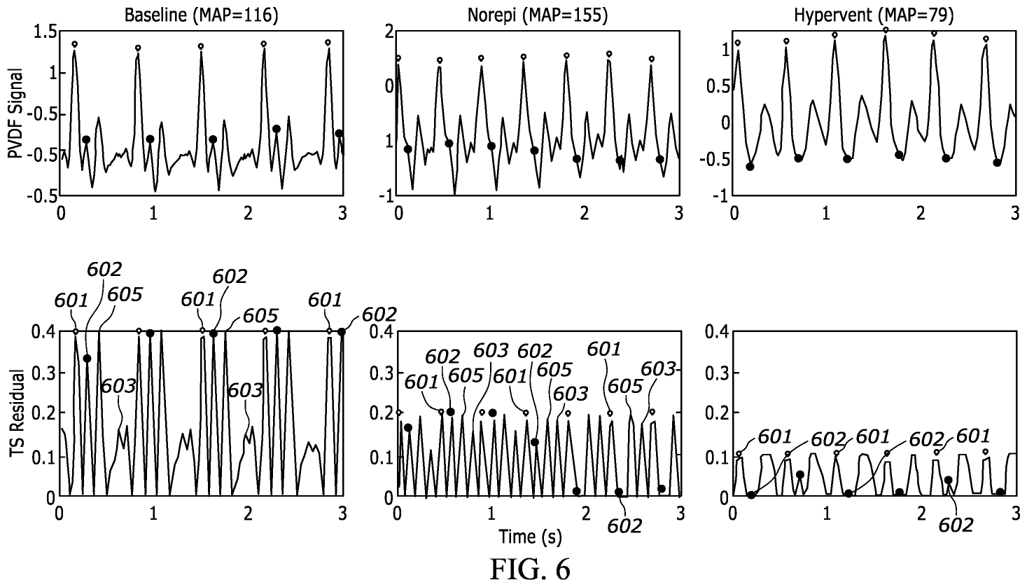

Four animal based tests were conducted where BP was modulated in a swine model using norepinephrine infusions to increase BP, and hyperventilation to induce vasodilation to decrease BP. Additionally, normal saline was infused between maneuvers in order to test the proposed approach at different levels of blood volume and fluid responsiveness. FIG. 6 includes multiple plots of signals versus time during baseline and the norepinephrine and hyperventilation maneuvers. The waveforms presented in FIG. 6 were generated by a PVDF ring sensor that measured the pulses of swine. To demonstrate the potential for using the delay and relative amplitude of reflection waves in estimating BP, signal features were calculated from the waveforms of FIG. 6.

The features of the waveforms were processed by applying a taut string approximation to the waveforms. The taut string approximation produces an outline of the signal and removes low amplitude fluctuations. By subtracting the taut string approximation from the original signal, the low amplitude features of the signal that are associated with reflection waves are essentially amplified, as shown in the bottom row of FIG. 6. The open circles in FIG. 6 correspond to the incident wave peaks 601, while the closed circles correspond to the first reflected wave peak amplitudes 602, similar to the first reflected wave peaks 202 in FIG. 3. The top row of FIG. 6 shows the original PVDF waveforms and the bottom row shows the same corresponding waveforms after the taut string approximation is subtracted. Subtraction of the taut string approximation allows for identification of the reflected waves and their peak amplitudes 602 and 603. For example, the first reflected waves are not visible in the original PVDF waveforms (top row) during norepinephrine infusion (middle column) and hyperventilation (right column). However, after the taut string approximation is subtracted from the original PVDF waveforms (bottom row), the reflected waves can be identified. The time delay between the incident wave peaks 601 and reflected wave peaks 602 can be calculated to determine the pulse wave velocity (i.e., the PWV of EQ. 4). In addition, the reflected wave amplitude peaks 602 may be measured and used to estimate the reflection coefficient and the vascular resistance, r of EQ. 4. The reflection coefficient and vascular resistance may then be used to estimate BP using a regression model. Note, in the bottom row of FIG. 6, that the second peak 605 and third peak 603, when visible, correspond respectively to the dicrotic notch and the second reflected wave. The second reflected wave can also be used to estimate BP in the same manner as described above that uses the first reflection wave.

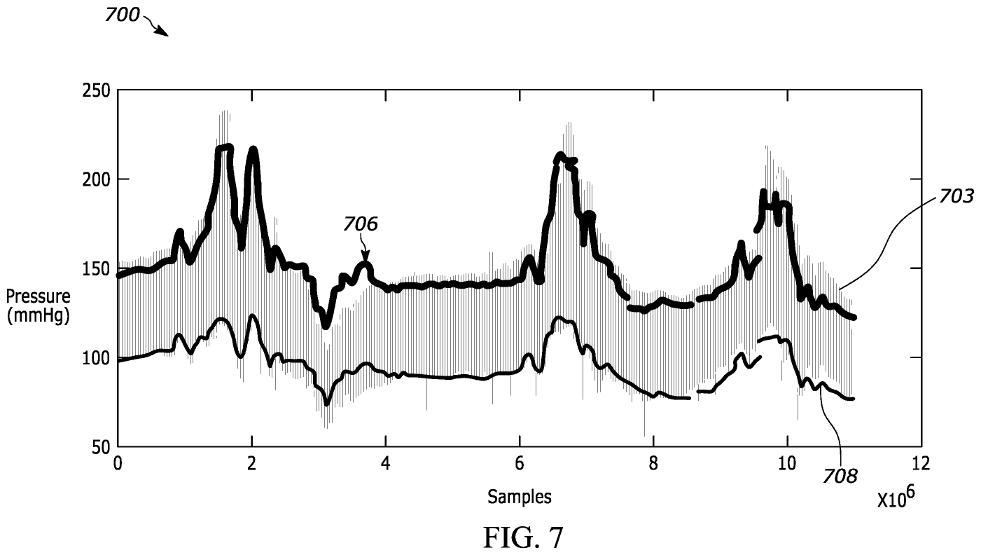

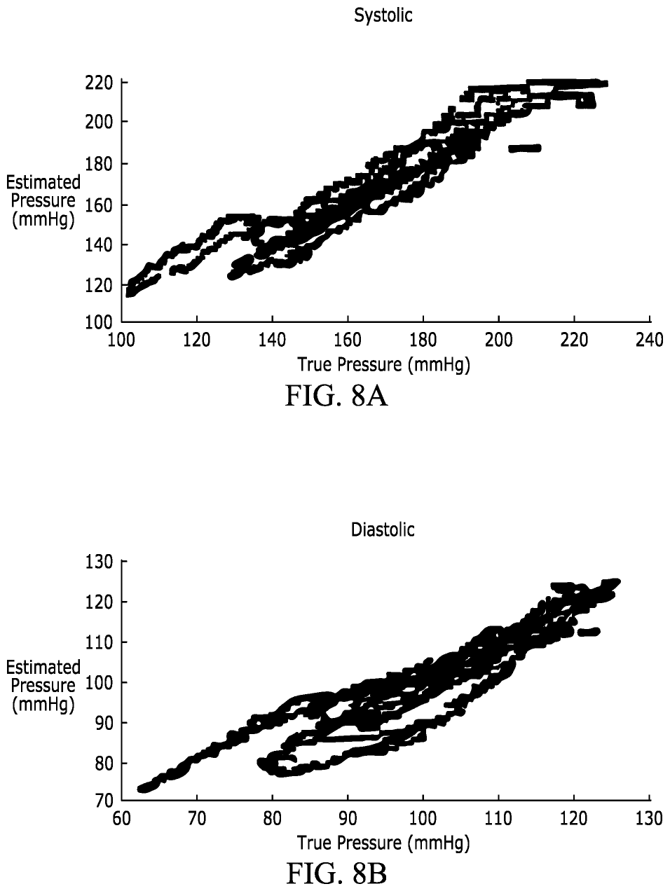

FIG. 7 is a plot 700 of the arterial BP showing the systolic and diastolic arterial BP for different sample times represented, respectively, by the upper and lower boundaries of the data band 703. The plot 700 further presents estimated systolic pulse data 706 and diastolic pulse data 708, using the taut string approach described above. As previously discussed, invasively measured arterial BP is the clinical gold-standard for BP measurement. FIGS. 8A and 8B are plots of systolic and diastolic BP, respectively, as measured by the disclosed methods versus the BP as measured by the invasively measured arterial BP. The average correlation between the estimated and true values across the measurements was 0.93 for both the systolic and diastolic pressures showing a high level of accuracy of the disclosed measurement devices and methods.

The ability to monitor hemodynamic variables such as BP and VR continuously during daily activity may have an enormous impact on patient care and experience. Cuff-based BP monitors are bulky and obtrusive, work non-continuously and are inaccurate. The disclosed devices and methods allow for reliable and convenient on-demand and/or continuous monitoring of hemodynamic variables such as BP, CO and VR. The disclosed systems and methods may allow for the identification and monitoring of a multitude of health conditions including stroke, heart failure, hypertension, trauma, sepsis, diabetes, and others. Further, the described devices and methods have been used to identify patients with low cardiac indices. The PVDF sensor was used to monitor patients undergoing cardiac catheterization. The cardiac index was measured using invasive techniques that are used in clinical practice. A receiver operating characteristic curve was generated and an area of 0.83 under the curve was achieved, indicating that the disclosed methods are effective for stratification of patients with low cardiac indices.

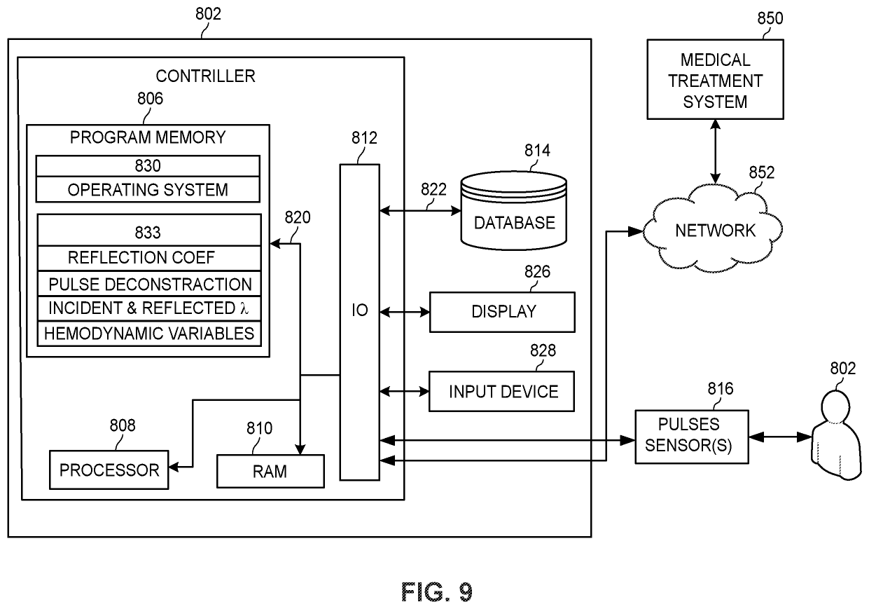

FIG. 9 is an example block diagram of an example hemodynamic measurement system 800, illustrating the various components used in implementing an example embodiment of processes described herein. A signal-processing device 802 (or “signal processor”) may be configured to examine a patient or healthy subject 820 via one or more sensors 816 in accordance with executing the functions of the disclosed embodiments. The sensor 816 may be one or more wearable sensors such as the ring sensor 100, for example. The sensor 816 may be any sensor capable of capturing pulses in accordance with the method 400, for example, whether wearable or otherwise attachable to a subject. The signal-processing device 802 may have a controller 804 operatively connected to a database 814 via a link 822 connected to an input/output (I/O) circuit 812. It should be noted that, while not shown, additional databases may be linked to the controller 804 in a known manner. The controller 804 includes a computer-readable media such as program memory 806, one or more processors 808 (may be called microcontrollers or a microprocessors), a random-access memory (RAM) 810, and the input/output (I/O) circuit 812, all of which are interconnected via an address/data bus 820. It should be appreciated that although only one processor 808 is shown, the controller 804 may include multiple microprocessors 808. Similarly, the memory of the controller 804 may include multiple RAMs 810 and multiple program memories 806. Although the I/O circuit 812 is shown as a single block, it should be appreciated that the I/O circuit 812 may include a number of different types of I/O circuits. The RAM(s) 810 and the program memories 806 may be implemented as semiconductor memories, magnetically readable memories, and/or optically readable memories, for example.

The computer-readable media 806 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units 808 of the computing device 802 may represent a CPU-type processing unit, a GPU-type processing unit, a field-programmable gate array (FPGA), another class of digital signal processor (DSP), or other hardware logic components that can be driven by a CPU.

A link 824, which may include one or more wired and/or wireless (Bluetooth, WLAN, etc.) connections, may operatively connect the controller 804 to the sensor(s) 816 through the I/O circuit 812. As a wearable sensor, the sensor 816 may be operatively connected to the person 820 at a location corresponding to a sampling region of the person in accordance with techniques herein, including but not limited to a person's wrist, finger, arm, ankle, waist, leg, or another part of the body for measuring the pulse of the person.

The program memory 806 and/or the RAM 810 may store various applications (i.e., machine readable instructions) for execution by the processor 808. For example, an operating system 830 may generally control the operation of the signal-processing device 802 and provide a user interface to the signal-processing device 802 to implement the processes described herein. The program memory 806 and/or the RAM 810 may also store a variety of subroutines 832 for accessing specific functions of the signal-processing device 802. By way of example, and without limitation, the subroutines 832 may include, among other things: a subroutine for determining the BP of the person from the reflection coefficient and the delay between the incident and reflection waves; a subroutine for deconstructing the pulse waveform to determine an incident pulse waveform and a reflected pulse waveform; a subroutine for determining an amplitude of the reflected pulse waveform; a subroutine for determining a reflection coefficient from the obtained pulse waveform; a subroutine for determining a time delay of the reflected pulse waveform; a subroutine for determining the BP of the person from the amplitude of the reflected pulse waveform; a subroutine for determining a VR of the person; and/or a subroutine determining a CO of the person. The subroutines 832 may implement any of the example methods described and illustrated herein to determine hemodynamic variables such as BP from captured sensor signals. These subroutines may be executed through one or more apps executed on the signal-processing device 802. The one or more apps may include a GUI interface for interaction by a user via the display screen 826. In the illustrated example, the subroutines 832 include performing pulse deconstruction, determining an incident and reflected wave, determining a reflection coefficient, and determining hemodynamic variables of the subject 820.

The subroutines 832 may also include other subroutines, for example, implementing software keyboard functionality, haptic touchscreen functionality, and/or interfacing with other hardware in the signal-processing device 802, etc. The program memory 806 and/or the RAM 810 may further store data related to the configuration and/or operation of the signal-processing device 802, and/or related to the operation of the one or more subroutines 832. For example, sensor data such as a pulse of a person may be data gathered by the sensor 816, data determined and/or calculated by the processor 808, etc. In addition to the controller 804, the signal-processing device 802 may include other hardware resources. The signal-processing device 802 may also include various types of input/output hardware such as a visual display 826 and input device(s) 828 (e.g., keypad, keyboard, etc.). In an embodiment, the display 826 is touch-sensitive and may cooperate with a software keyboard routine as one of the software routines 832 to accept user input.

It may be advantageous for the signal-processing device 802 to communicate with a broader medical treatment system 850 through a network 852, using any of a number of known networking devices and techniques (e.g., through a computer network such as a hospital or clinic intranet, the Internet, etc.). For example, the system may be connected to a medical records database, hospital management processing system, health care professional terminals (e.g., doctor stations, nurse stations), person monitoring systems, automated drug delivery systems such as smart pumps, smart infusion systems, automated drug delivery systems, etc. Accordingly, the disclosed embodiments may be used as part of an automated closed loop system or as part of a decision assist system.

The network 852 may be a public network such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 852 can include a number of devices that facilitate network communications and/or form a hardware basis for the networks, such as switches, routers, gateways, access points (such as a wireless access point as shown), firewalls, base stations, repeaters, backbone devices, etc.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion such as a Contrast Agent Injection System shown in FIG. 3) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed is:

1. A device for non-invasively measuring hemodynamic variables, the device comprising:
a sensor configured to sense a pulse of a person and to generate signals indicative of the pulse of the person;
a support structure physically coupled to the sensor to physically support the sensor during operation of the sensor; and
a processor in communication with the sensor configured to:
collect the signals indicative of the pulse of the person from the sensor,
determine, from the signals, a waveform representative of the pulse,
perform signal processing on the waveform to identify features of the waveform, and
perform processing on the features to determine a hemodynamic variable of the person.

2. The device of claim 1, wherein the processor is further configured to deconstruct a waveform to determine an incident pulse waveform and a reflected waveform.

3. The device of claim 2, wherein to identify features of the waveform, the processor is further configured to:
determine an amplitude of the incident pulse waveform
determine an amplitude of the reflected pulse waveform;
determine a time delay of the reflected pulse waveform; and
determine the hemodynamic variable of the person from the amplitude of the incident waveform, amplitude of the reflected waveform, and the time delay of the reflected pulse waveform.

4. The device of claim 2, wherein the processor is further configured to identify multiple reflection waves and to analyze features of the multiple reflection waves to determine at least one hemodynamic variable of the hemodynamic variables.

5. The device of claim 2, wherein the hemodynamic variable comprises blood pressure.

6. The device of claim 1, wherein the sensor comprises a polyvinylidene fluoride sensor.

7. The device of claim 1, wherein the hemodynamic variable comprises vascular resistance, and wherein the processor is further configured to:
determine (i) a time delay between the incident waveform and the reflected waveform, (ii) an amplitude of the incident wave, and (iii) an amplitude of the reflected wave,
determine, from the time delay, a pulse wave velocity,
determine, from the amplitude of the incident wave and the amplitude of the reflected wave, a reflection coefficient,
determine, from the reflection coefficient, an effective radius of arteries of the person, and
determine, from the pulse wave velocity and the effective radius of the arteries, a vascular resistance of the person.

8. The device of claim 7, wherein the hemodynamic variable comprises cardiac output, and wherein the processor is further configured to:
determine, from the pulse wave velocity and the effective radius of the arteries, a blood pressure of the person, and
determine, from the blood pressure and the vascular resistance, a cardiac output of the person.

9. The device of claim 2, wherein the processor is further configured to: determine, from the incident wave and the reflected wave, a transfer function representing the mechanical dynamics of the arterial system of the person.

10. The device of claim 1, wherein the processor is further configured to determine variables characterizing the mechanical function of arteries and the cardiovascular system.

11. The device of claim 1, wherein the processor is further configured to determine components of systemic vascular resistance.

12. The device of claim 1, wherein the sensor is selected from the group consisting of: an optical sensor, a force based sensor, an electrical based sensor, and an ultrasonic sensor.

13. The device of claim 1, wherein the sensor comprises a plurality of sensors.

14. The device of claim 1, wherein the support structure is a band.

15. The device of claim 1, wherein the sensor is configured to sense a pulse of a person from a body part selected from the group consisting of: a finger, an arm, a thigh, an ankle, a toe, a temple, a nose, a chest, and a neck.

16. The device of claim 1, wherein the hemodynamic variables include a variable selected from the group consisting of: blood pressure, cardiac output, and vascular resistance.

17. A method for noninvasive measurement of hemodynamic variables, the method comprising:
physically configuring a sensor to measure the pulse of a person;
generating, by the sensor, a pulse waveform indicative of the pulse of the person;
obtaining, by a processor, the pulse waveform from the sensor;
determining, by the processor, a reflection coefficient and a delay between an incident wave and reflected wave from the obtained pulse waveform; and
determining, by the processor, the hemodynamic variables of the person from the reflection coefficient.

18. The method of claim 17, further comprising deconstructing, by the processor, the pulse waveform to determine an incident pulse waveform and a reflected pulse waveform.

19. The method of claim 18, further comprising:
determining, by the processor, an amplitude of the reflected pulse waveform;
determining, by the processor, a time delay of the reflected pulse waveform; and
determining, by the processor, the blood pressure of the person from the amplitude and time delay of the reflected pulse waveform.

20. The method of claim 18, further comprising:
identifying, by the processor, multiple reflection waves from the reflected pulse waveform; and
analyzing, by the processor, features of the multiple reflection waves to determine at least one hemodynamic variable of the hemodynamic variables.

21. The method of claim 17, wherein the sensor comprises a polyvinylidene fluoride sensor.

22. The method of claim 17, further comprising determining, by the processor, a cardiac output of the person.

23. The method of claim 17, further comprising determining, by the processor, a vascular resistance of the person.

24. The method of claim 17, further comprising determining, by the processor, a transfer function for the arterial system of the person.

25. The method of claim 17, further comprising determining, by the processor, variables characterizing the mechanical function of arteries and the cardiovascular system.

26. The method of claim 17, further comprising determining, by the processor, components of systemic vascular resistance.

27. The method of claim 17, wherein the sensor is selected from the group consisting of: an optical sensor, a force based sensor, an electrical based sensor, and an ultrasonic sensor.

28. The method of claim 17, wherein the hemodynamic variables includes a variable selected from the group consisting of: blood pressure, cardiac output, and vascular resistance.

29. A device for non-invasively measuring hemodynamic variables, the device comprising:
a sensor configured to sense a pulse of a person and to generate signals indicative of the pulse of the person;
a support structure physically coupled to the sensor to physically support the sensor during operation of the sensor; and
a processor in communication with the sensor configured to:
collect the signals indicative of the pulse of the person from the sensor,
determine, from the signals, a waveform representative of the pulse,
deconstruct the waveform to determine an incident pulse waveform and a reflected pulse waveform,
determine an amplitude of the reflected pulse waveform;
determine a time delay of the reflected pulse waveform; and
determine a hemodynamic variable of the person from the amplitude and time delay of the reflected pulse waveform, wherein the hemodynamic variables includes at least one of blood pressure, cardiac output, and vascular resistance.
